(19) United States
(12) Patent Application Publication
(10) Pub. No.: US 2007/0093823 A1
(43) Pub. Date: Apr. 26, 2007

(54) Spinal distraction device and methods of manufacture and use

(75) Inventors: Kevin C. Booth, Pleasanton, CA (US); Joseph M. Grant, Pleasanton, CA (US)

Correspondence Address: LUCE, FORWARD, HAMILTON & SCRIPPS LLP, 11988 EL CAMINO REAL, SUITE 200, SAN DIEGO, CA, 92130, US

(73) Assignee: NuVasive, Inc., San Diego, CA (US)

(21) Appl. No.: 11/529,936

(22) Filed: Sep. 29, 2006

Related U.S. Application Data
(60) Provisional application No. 60/722,208, filed on Sep. 29, 2005.

(51) Int. Cl. A61F 2/30

(52) U.S. Cl. 606/61

(57) ABSTRACT

A spinal distraction device is provided having a generally H-shaped cross section that maintains a desired space between adjacent spinous processes. The distraction device includes a body and two pairs of parallel sidewalls. The sidewalls create channels for receiving respective spinous processes. The distraction device is fused to the spinous processes with fixation devices.

### CROSS-REFERENCE TO RELATED APPLICATION

This application claims priority to U.S. Provisional Patent Application Ser. No. 60/722,208, filed Sep. 29, 2005, which is hereby incorporated in its entirety by reference herein.

### FIELD OF THE INVENTION

The present invention relates to devices for improving the spinal stability of a patient. More particularly, the present invention relates to a spinal distraction device that may be implanted between adjacent spinous processes and is particularly well suited for percutaneous delivery.

### BACKGROUND OF THE INVENTION

As people age, they often experience degenerative conditions affecting the spinal structures. The conditions often cause pain and discomfort that may, in severe cases, be debilitating. Those conditions may include, for example, spinal stenosis, facet arthropathy and degenerative disc disease.

Spinal stenosis is the thickening of the bones that make up the spine. That thickening often reduces the size of the passageways through which the spinal column, nerves and blood vessels pass. Those passageways include the central spinal canal and lateral foramena. In some cases, the space may be reduced to such a degree that the spinal column, nerves and/or blood vessels become impinged, or compressed, causing pain and/or numbness.

Facet arthropathy (degeneration and arthritis of the facet joints) and degenerative disc disease often contribute to the instability of the spine. That instability may exacerbate or independently cause compression of the spinal cord, nerves and/or blood vessels within and surrounding the spine.

The methods used to treat those spine conditions vary depending on the severity. For example, in less severe cases, non-surgical methods such as modifying activities or administering anti-inflammatory medications may be utilized. However, treatment of more severe cases often includes major surgical procedures such as spinal decompression surgery.

A common problem with such procedures is that they are very invasive and require the severing of tissues surrounding the vertebral bodies causing the compression. As a result, the patient is oftentimes subjected to lengthy rehabilitation. In addition, some patients, especially elderly patients, may not have the stamina to endure such invasive procedures or lengthy rehabilitation.

Other efforts have focused on creating distraction devices for maintaining a desired space between adjacent spinous processes. One such example of a distraction device is described in U.S. Pat. No. 6,699,247 to Zucherman et al. The distraction device described in that patent includes an expandable structure that includes two saddles facing opposite directions. The opening of each saddle is oriented to face a spinous process and the device is expanded to increase the space between the spinous processes. The saddles may be tethered to the spinous processes to inhibit migration of the device while allowing relative motion between each spinous process and the associated saddle and between the spinous processes. The device is also constructed from a flexible material to allow for that relative motion.

A disadvantage of the device is that where fusion between the spinous processes is desired, the device is inadequate because it is designed and constructed so that there is relative movement.

### SUMMARY OF THE INVENTION

In view of the foregoing, it is an object of the present invention to provide a spinal distraction device that overcomes the drawbacks of previously known treatments and devices, and which may be implanted using open surgical, minimally invasive or percutaneous implantation techniques.

It is also an object of the present invention to provide a percutaneously deliverable spinal distraction device that may be constructed from bone allograft.

It is a further object of the present invention to provide a spinal distraction device that allows for fusion of the device with the adjacent spinous processes.

These and other objects of the present invention are accomplished by providing a spinal distraction device constructed from bone allograft. The device has an H-shaped cross-section that provides channels, each of which is designed to receive a spinous process. The device also includes apertures for receiving fixation devices, such as screws, sutures or wire.

In one embodiment, the distraction device includes a body portion and two pairs of parallel side walls. The side walls create channels for receiving the adjacent spinous processes. Apertures are also provided through the side walls for receiving fixation screws.

In another embodiment, the distraction device includes a body portion and two pairs of parallel side walls, the side walls create channels for receiving the adjacent spinous processes. In this embodiment, an aperture is provided through the body portion for receiving a suture that is used to fuse the device within the space between the adjacent spinous processes.

### BRIEF DESCRIPTION OF THE DRAWINGS

The above and other objects and advantages of the present invention will be apparent upon consideration of the following detailed description, taken in conjunction with the accompanying drawings, in which like reference numerals refer to like parts throughout, and in which:

FIG. 1 is a side view of an exemplary spinal distraction device of the present invention implanted between adjacent spinous processes;

FIG. 2 is a perspective view of the spinal distraction device of FIG. 1;

FIG. 3 is a side view of the spinal distraction device of FIG. 1;

FIG. 4 is another side view of the spinal distraction device of FIG. 1;

FIG. 5 is a side view of another exemplary embodiment of a spinal distraction device of the present invention implanted between adjacent spinous processes;

FIG. 6 is a perspective view of the spinal distraction device of FIG. 5;

FIG. 7 is a side view of the spinal distraction device of FIG. 5; and

FIG. 8 is another side view of the spinal distraction device of FIG. 5.

### DETAILED DESCRIPTION OF THE INVENTION

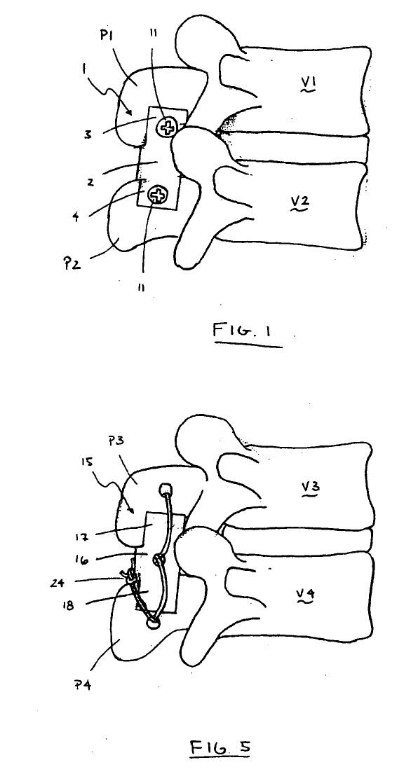

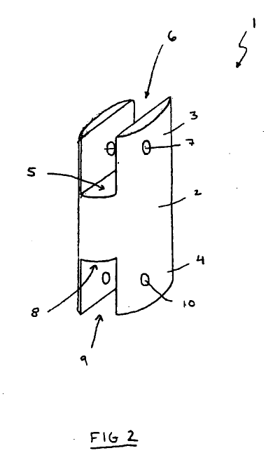

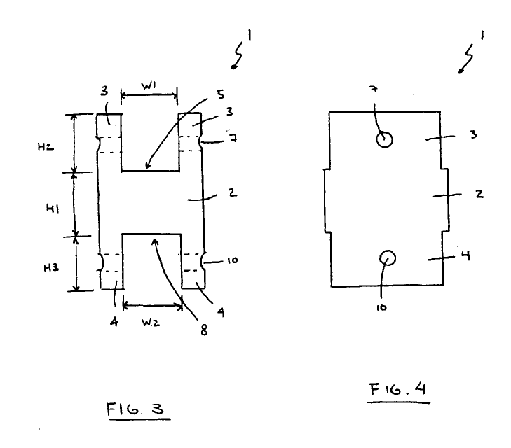

The present invention is directed to a spinal distraction device 1 that is used to maintain a desired space between adjacent spinous processes, as shown in FIG. 1-4. Distraction device 1 may be implanted between adjacent vertebrae V1 and V2 to maintain a desired space between the vertebrae. In particular, the distance between a superior spinous process P1 of a superior, or upper, vertebra V1 and an inferior spinous process P2 of inferior, or lower, vertebra V2 is maintained by distraction device 1.

Distraction device 1 is generally H-shaped and generally includes a main body portion 2, a pair of superior side walls 3, and a pair of inferior side walls 4. Body portion 2 is a generally columnar member that has a height H1 corresponding to the desired space between adjacent spinous processes. As shown, body portion 2 has an oval cross-section, but it shall be appreciated that body portion 2 may have any cross-sectional shape, such as a rectangle, circle or semi-circle and body portion 2 may be tubular.

Side walls 3 extend upward from a top surface 5 of body portion 2. Side walls 3 are generally parallel and spaced from each other by a distance W1. Side walls 3 and top surface 5 combine to define a superior channel 6 having a width corresponding to distance W1.

One or more apertures 7 may be provided through superior side walls 3. Apertures 7 are configured to receive fixation devices, such as screws or sutures, for the purpose of fusing side walls 3 to the adjacent spinous process P1. It shall be appreciated that one aperture 7 may be provided that extends through only one of side walls 3. In addition, although apertures 7 are shown generally aligned, it shall be appreciated that apertures 7 need not be aligned and may be offset so that multiple fixation devices may be inserted through side walls 3.

Side walls 4 extend downward from a bottom surface 8 of body portion 2. Side walls 4 are generally parallel and spaced from each other by a distance W2. In addition, side walls 4 are generally parallel to side walls 3 such that distraction device 1 has an H-shaped cross-section, as shown in FIG. 3. Side walls 4 and bottom surface 8 together define an inferior channel 9 having a width W2.

Apertures 10 may also be provided through side walls 4. Similar to apertures 7, apertures 10 are configured to receive fixation devices, such as screws or sutures, for the purpose of fusing side walls 4 to the adjacent spinous process P2. It shall be appreciated that one aperture 10 may be provided that only extends through one of side walls 4. In addition, although apertures 10 are shown generally aligned, it shall be appreciated that apertures 10 may be offset so that multiple fixation devices may be inserted through side walls 4.

Superior channel 6 is configured to receive a lower portion of spinous process P1. In particular, distance W1 approximates the width of spinous process P1 so that there is limited relative lateral motion between side walls 3 and spinous process P1. In addition, side walls 3 have a height H2 sufficient for fixation of side walls 3 with spinous process P1 by fixation screws 11 that extend through apertures 7.

Similarly, inferior channel 9 is configured to receive an upper portion of spinous process P2. Distance W2 approximates the width of spinous process P2 so that there is limited relative lateral motion between side walls 4 and spinous process P2. Furthermore, side walls 4 have a height H3 sufficient for fixation of side walls 4 with spinous process P2 by fixation screws 11 that extend through apertures 10. It shall be appreciated that fixation screws 11 may be any fixation screw known in the art.

Distraction device 1 is formed from a material that is preferably osteoconductive and/or osteoinductive, such as bone allograft. The material choice helps to ensure fusion between distraction device 1 and the adjacent spinous processes P1 and P2. Distraction device 1 may be formed from the allograft material and packaged by any method known in the art. For example, the device may be kept frozen or freeze-dried until just prior to its implantation.

In order to implant distraction device 1, access to superior spinous process P1 and inferior spinous process P2 is provided, for example using percutaneous surgical procedures known in the art. Thereafter, spinous process P1 is distracted from spinous process P2. Next, distraction device 1 is introduced, for example via a cannula, into the space between spinous process P1 and spinous process P2. Distraction device 1 is oriented such that superior spinous process P1 is located within superior channel 6 and inferior spinous process P2 is located within inferior channel 9. After distraction device 1 is properly located, side walls 3 are fused to superior spinous process P1 and side walls 4 are fused to inferior spinous process P2 by implanting fixation screws 11 through apertures 7 and 10, respectively.

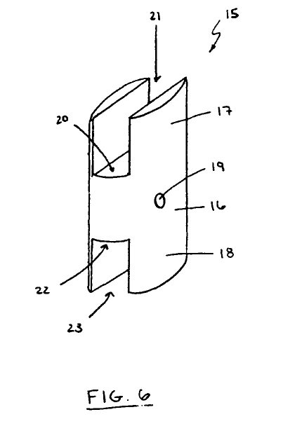

Another embodiment of a spinal distraction device 15 is shown in FIGS. 5-8. Distraction device 15 may be implanted between adjacent vertebrae V3 and V4 to maintain a desired space between the vertebrae, as shown in FIG. 5, and as described with respect to the previous embodiment above. Particularly, distraction device 15 maintains a space between a superior spinous process P3 of a superior vertebra V3 and an inferior spinous process P4 of an inferior vertebra V4 in a patient's spine.

Similar to the previously described embodiment, distraction device 15 generally includes a body portion 16, a pair of superior side walls 17, and a pair of inferior side walls 18. Body portion 16 is a generally columnar member that has a height H4 corresponding to the desired space between adjacent spinous processes. Body portion 16 may have any cross-sectional shape, such as a rectangle, circle or semi-circle and body portion 16 may be tubular.

An aperture 19 extending through body portion 16 may also be provided for fixing distraction device 15 to adjacent spinous processes with a suture, or wire, as will be described in greater detail below.

Side walls 17 extend upward from a top surface 20 of body portion 16. Side walls 17 are generally parallel and spaced from each other to form a superior channel 21 having a width W3.

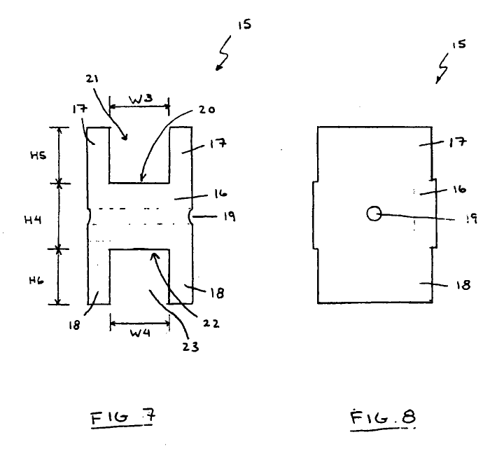

Side walls 18 extend downward from a bottom surface 22 of body portion 16. Side walls 18 are generally parallel and spaced from each other to form an inferior channel 23 having a width W4. In addition, side walls 18 are generally parallel to side walls 17 such that distraction device 15 has an H-shaped cross-section, as shown in FIG. 7.

Superior channel 21 is configured to receive a lower portion of spinous process P3. In particular, distance W3 approximates the width of spinous process P3 so that there is limited relative lateral motion between side walls 17 and spinous process P3. In addition, side walls 17 have a height H5 sufficient to restrict lateral movement of distraction device 15 with respect to spinous process P3.

Similarly, inferior channel 23 is configured to receive an upper portion of spinous process P4. Distance W4 approximates the width of spinous process P4 so that there is limited relative lateral motion between side walls 18 and spinous process P4. Furthermore, side walls 18 have a height H6 sufficient to restrict lateral movement of distraction device 15 with respect to spinous process P4.

Distraction device 15 may be introduced as described above with respect to the previous embodiment. After introduction, a suture, or wire, may be used to retain distraction device 15 in the space between superior spinous process P3 and inferior spinous process P4. The suture may be woven through aperture 19 and additional apertures A3 and A4 drilled through spinous process P3 and spinous process P4, respectively, as shown in FIG. 5. After the suture is woven through he apertures, they may be tied to secure the device. Alternatively, the suture may be woven through aperture 19 and over, or through, other vertebral features, thereby obviating the need for apertures A3 and/or A4.

Distraction device 15 is preferably formed from a material that is osteoconductive and/or osteoinductive, such as bone allograft. The material therefore promotes fusion between distraction device 15 and the adjacent spinous processes. Distraction device 15 may be shaped from a piece of bone allograft and stored frozen or freeze-dried as is well-known in the art.

An advantage of distraction device 15 is that a smaller piece of allograft may be used because the side walls do not have to be long enough to support screw fixation.

While preferred embodiments of the invention are described above, it will be apparent to one skilled in the art that various changes and modifications may be made.

What is claimed is:

1. A spinal distraction device, comprising:
a body portion including a top surface and a bottom surface;
a superior side wall extending from the top surface, wherein the superior side wall includes a transverse aperture; and
an inferior side wall extending from the bottom surface, wherein the inferior side wall includes a transverse aperture.

2. The spinal distraction device of claim 1, wherein the transverse aperture of the superior side wall is configured to receive a screw.

3. The spinal distraction device of claim 1, wherein the transverse aperture of the inferior side wall is configured to receive a screw.

4. The spinal distraction device of claim 1, wherein the transverse aperture of the superior side wall is configured to receive a suture.

5. The spinal distraction device of claim 1, wherein the transverse aperture of the inferior side wall is configured to receive a suture.

6. The spinal distraction device of claim 1, wherein the body portion, superior side wall and inferior side wall are monolithic.

7. The spinal distraction device of claim 6, wherein the body portion, superior side wall and inferior side wall are bone allograft.

8. The spinal distraction device of claim 1, further comprising a second superior side wall extending from the top surface of the body portion, wherein the second superior side wall is parallel to the first superior side wall.

9. The spinal distraction device of claim 8, wherein the second superior side wall includes a transverse aperture.

10. The spinal distraction device of claim 9, wherein the transverse aperture of the second superior side wall is aligned with the transverse aperture of the first superior side wall.

11. The spinal distraction device of claim 1, further comprising a second inferior side wall extending from the bottom surface of the body portion, wherein the second inferior side wall is parallel to the first inferior side wall.

12. The spinal distraction device of claim 11, wherein the second inferior side wall includes a transverse aperture.

13. The spinal distraction device of claim 12, wherein the transverse aperture of the second inferior side wall is aligned with the transverse aperture of the first inferior side wall.

14. A spinal distraction kit, comprising:
a spinal distraction device, comprising a body portion including a top surface and a bottom surface; a pair of parallel superior side walls extending from the top surface, wherein at least one superior side wall includes a transverse aperture; and a pair of parallel inferior side walls extending from the bottom surface, wherein at least one inferior side wall includes a transverse aperture; and
a plurality of fixation devices.

15. The spinal distraction kit of claim 14, wherein at least one fixation device is a screw.

16. The spinal distraction kit of claim 14, wherein at least one fixation device is a suture.

17. A method of performing spinal distraction, comprising:
accessing a superior spinous process and an inferior spinous process adjacent a spinal disc;
distracting the superior spinous process relative to the inferior spinous process;
implanting a distraction device between the superior spinous process and the inferior spinous process, the distraction device comprising a body portion including a top surface and a bottom surface; a pair of parallel superior side walls extending from the top surface, wherein at least one superior side wall includes a transverse aperture; and a pair of parallel inferior side walls extending from the bottom surface, wherein at least one inferior side wall includes a transverse aperture, wherein the distraction device is configured so that the superior spinous process is at least partially disposed between the superior side walls and the inferior spinous process is at least partially disposed between the inferior side walls; and
fixing the superior side walls to the superior spinous process and the inferior side walls to the inferior spinous process.

18. The method of performing spinal distraction of claim 17, wherein the step of accessing is by percutaneous surgical procedures.

19. The method of performing spinal distraction of claim 17, wherein the step of fixing is performed by inserting screws through the side wall apertures and the corresponding spinous processes.

20. The method of performing spinal distraction of claim 17, wherein the step of fixing is performed by inserting sutures through the side wall apertures and the corresponding spinous processes.
